(19) United States
(12) Patent Application Publication
(10) Pub. No.: US 2010/0069784 A1
(43) Pub. Date: Mar. 18, 2010

(54) Urological medical device and method for analyzing urethral properties

(76) Inventor: Jerry G. Blaivas, Pound Ridge, NY (US)

Correspondence Address: HOFFMAN WARNICK LLC, 75 STATE STREET, 14TH FLOOR, ALBANY, NY, 12207, US

(21) Appl. No.: 12/283,948

(22) Filed: Sep. 16, 2008

(51) Int. Cl. A61F 2/00; A61B 5/03

(52) U.S. Cl. 600/561; 600/29

(57) ABSTRACT

A highly flexible urological medical device for analyzing urethral properties and for optimally placing a periurethral injection and/or a support element, such as suburethral sling and/or a suspension suture, to treat urinary incontinence.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON COMPACT DISC

Not Applicable.

### BACKGROUND OF THE INVENTION

(1) Field of the Invention

The present invention generally relates to a urological medical device and a method for use of same.

(2) Description of Related Art

Urinary continence is normally maintained by the sphincteric function of the proximal two thirds of the female urethra and the prostatic urethra in men. The mechanism by which the sphincter functions is not completely understood. Normally, the sphincter remains closed at all times except during voluntary micturition. Even when there is an increase in abdominal pressure, such as during coughing, straining and physical activity, the sphincter remains closed and continence is maintained.

The female urethra is about 3 cm to 4 cm in length and the proximal two thirds of the urethra functions as the sphincter mechanism. However, there are no clear cut anatomic landmarks, either macroscopically or microscopically that delineate the sphincter. Rather, it is an admixture of smooth and striated muscle, extracellular matrix, a highly compliant vascular plexus and a mucosal seal that constitute the sphincter. In addition, the urethra and bladder are normally supported in their anatomic position by the pelvic floor muscles and fascia.

Normally, when there is an increase in abdominal pressure (Pabd), the proximal urethra is held in place by the pelvic floor support structures and Pabd is transmitted equally to the bladder and urethra, so there is no change in the urethral closure pressure (Pureclos). Pureclos is the difference between vesical and urethral pressure. In addition, during increases in Pabd, there is a reflex contraction of the urethral and pelvic floor muscles. The net effect is that Pure remains greater than vesical pressure and continence is maintained.

Urinary incontinence may be caused by a number of different abnormalities: 1) sphincteric deficiency (sphincteric incontinence), 2) detrusor overactivity, 3) mixed incontinence (sphincteric incontinence and detrusor overactivity), 4) stress hyperreflexia, 5) urinary fistula, and 6) ectopic ureter. It is usually possible to correctly diagnose these conditions clinically by history, physical examination, bladder diary and pad test. However, merely knowing the diagnosis only permits a general approach to treatment. By performing more sophisticated tests of bladder and urethral function, it is possible to individualize therapy in such a way as to enhance the likelihood of a successful treatment outcome.

Urinary fistula and ectopic ureter are usually adequately diagnosed based on history, examination and cystoscopy. There is a need, however, to further refine the diagnosis of the other types of incontinence in order to better understand the pathophysiology and to individualize treatment based on the pathophysiology. The first step in diagnosis is to distinguish sphincteric incontinence from detrusor overactivity. This is accomplished by history, examination with a full bladder, diary and pad test. The diagnosis may be confirmed by urodynamics testing.

There are a number of theoretic mechanisms that describe the pathophysiology of sphincteric incontinence that can be broadly categorized into 1) intrinsic sphincter deficiency (ISD), and 2) urethral support abnormalities. ISD may be caused by: 1) weakness of the intrinsic smooth muscles of the urethra, 2) scarring of the urethral wall that results in decreased urethral compliance, 3) loss of the mucosa seal and 4) loss of the vascular cushion. There are several existing techniques to assess weakness of the intrinsic urethral musculature including the leak point pressure, the static urethral pressure profile and the stress urethral pressure profile.

The leak point pressure is measured with a catheter, which extends through the urethra into the bladder. The bladder is filled through the urethral catheter and, at filling increments of about 100 ml to 150 ml the patient is asked to cough and bear down until bladder capacity is reached or leakage occurs. The lowest vesical pressure (Pves) that causes visual or radiologic leakage from the urethral meatus is termed the vesical or valsalva leak point pressure (VLPP). The lower the VLPP, the weaker the sphincter and vice versa. VLPP may range from 0 cm H₂0 to about 200 cm H₂0.

Existing devices used to measure VLLP involve the use of a stiff urethral catheter that cannot conform to the shape of the urethra and that deforms the urethra during increases in abdominal pressure. This may result in inaccurate measurement of VLPP. See, for example, U.S. Pat. No. 6,056,699.

The prior art devices are also lacking in that they do not measure urethral compliance in a physiologic manner and do not assess abnormalities of the mucosal seal.

Compliance refers to the relation between a change in volume to a change in pressure. As used herein, urethral compliance can be calculated as (V2−V1)/(Pure2−Pure1), where V1 is the resting volume of the urethra, V2 is the volume of the urethra during an increase in Pves, Pure1 is the resting urethral pressure, and Pure2 is the urethral pressure during the increase in Pves. The volume of the urethra, approximated as the shape of a cylinder, is expressed as V=πr²h, where r is the radius of the urethra and h is the length of the urethra over which the volume is measured.

Regnier et al. describe a rigid probe comprised of five vinyl catheters glued together end-to-end such that the outer diameter steps down from 10 mm to 1.6 mm (30F to 5F). See Regnier C. H., Susset J. G., Ghonium G. M. and Biancani P., A new catheter to measure urethral compliance in females: Normal values, J Urol. 129:1060-1062, 1983; and Susset J. G., Ghoniem G. M. and Regnier C. H., Abnormal urethral compliance in females diagnosis, results and treatment; Preliminary study, J Urol. 129:1063-1065, 1983. The catheter is inserted into the urethra incrementally such that the inner diameter of a portion of the urethra disposed over the largest inserted catheter portion is sequentially stretched from 5F to 25F as Pure is measured. A single side hole in each catheter is slowly perfused with saline to keep the urethral wall from occluding the side hole while pressure is being measured. Pure is measured for each of the five urethral diameters artificially generated by insertion of the catheter.

Lose et al. describe a probe to measure Pves, Pure and urethral cross sectional area over a distance of 2 mm. See Lose G., Colstrup H., Saksager K. and Kristensen J. K., New method for static and dynamic measurement of related values of cross-sectional area and pressure in the female urethra, Neurourol & Urodyn 6:465-476, 1988; Lose G. and Colstrup H., Mechanical properties of the urethra in healthy and stress incontinent females: Dynamic measurements in the resting urethra, J Urol. 144:1258-1262, 1990; and Lose G., Urethral pressure and power generation during coughing and voluntary contraction of the pelvic floor in females with genuine stress incontinence, BJU. 67:580-585, 1991. The probe includes three catheters. The outer polyolefine catheter has an outside diameter of 4.3 mm and an inflatable balloon to distend the urethra. An inner polyolefine catheter has an outside diameter of 2.5 mm and includes four platinum electrodes to estimate cross sectional area using the field gradient principle. The third catheter includes two micro-tip transducers for measuring Pves and Pure. The probe is manually positioned sequentially at different parts of the urethra (proximal, mid and distal) and the balloon is inflated to different cross sectional areas (CA) at different rates of inflation. Pressure and cross sectional area changes to coughing and voluntarily contracting the sphincter are measured. Considering the urethra to be a cylinder, they defined urethral compliance of a 1 cm segment to be the change in cross section area divided by the change in pressure. The inverse of compliance is referred to by Lose et al. as elastance.

Both Regnier et al. and Lose et al. change the urethral diameter by artificially stretching the urethra from the inside with a catheter or balloon and, hence, are not physiologic and may invoke reflex urethral contractions. In contrast, the present invention allows the patient's own urine flow to naturally circumferentially expand the urethra. Further, neither the Regnier et al. nor the Lose probe are capable of (i) measuring the relevant physiologic properties at more than one place in the urethra at a time or (ii) measuring changes due to urine entering the urethra.

Existing devices are also deficient in that they do not properly assess urethral mobility. Abnormalities of urethral support have been attributed to weakness of the pelvic floor muscles and fascia, primarily subsequent to pregnancy and childbirth. Loss of pelvic floor support may have a number of different effects on urethral anatomy and function. Firstly, there may be rotational descent of the proximal urethra (range 0° to 75°), but no incontinence because the urethral walls remained coapted. Secondly, there may rotational descent of the urethra such that the urethra is pulled open because the anterior portion of the urethral wall is supported relatively better than the posterior portion. Thirdly, there may be no rotation at all, but during increased abdominal pressure, the urethra may widen and shorten. The latter two conditions result in incontinence.

The classic methods of assessing urethral mobility are the Q-tip angle and the chain cystogram, but both techniques only measure urethral angle and descent and do not assess the relationship between the two parameters, i.e., the urethral angle and concomitant urethral pressure at which urine leakage occurs, nor do they assess urethral shape or compliance, or define the axis around which the urethra rotates.

### BRIEF SUMMARY OF THE INVENTION

The present invention involves a novel urological medical device, e.g., for assessing urethral properties, and novel surgical methods for treating incontinence.

A urological medical device according to an exemplary embodiment of the present invention includes an elongate body adapted to be inserted into the urethra of a patient, the body is highly flexible along its length at normal body temperatures.

In an exemplary embodiment, the elongate body is made from an elastomeric material, e.g., having a durometer of less than 40 or 30 or 20 or 10 or 5 or 1 on the Shore A scale at body temperatures.

In an exemplary embodiment, the elongate body includes a lumen extending longitudinally along an entire length of the elongate member and the body has a radial wall thickness of less than or equal to approximately 100 mils or 75 mils or 50 mils or 25 mils or 15 mils. In an exemplary embodiment, the body has a radial wall thickness in the range of 2 to 10 mils.

In an exemplary embodiment, the elongate body includes a lumen and is adapted to stretch circumferentially upon application of a pressure in the lumen greater than approximately 5 cm H₂0.

In an exemplary embodiment, the urological medical device further includes a plurality of tracking members fixed to the elongate body, and a locating device configured to track the position of the tracking members.

In an exemplary embodiment, the urological medical device includes one or more pressure sensors connected to the elongate body. The pressure sensors may serve as the tracking members.

In an exemplary embodiment, the urological medical device may include one or more groups of two or more pressure sensors. Each of the two or more pressure sensors are connected to the elongate body at the same point along a length of the elongate body but are spaced apart from each other circumferentially.

In an exemplary embodiment, each of the one or more groups are spaced apart from each other along the length of the elongate body.

In an exemplary embodiment, the urological medical device includes a retention element on one end of the elongate body configured to removably retain at least a portion of the elongate body in the urethra.

In an exemplary embodiment, the elongate body includes a proximal tip having a lumen adapted to receive a proximal end of an insertion element used to insert the elongate body into the urethra of a patient.

In an exemplary embodiment, the elongate body has a length greater than approximately 30 cm and does not have a retention means used to removably retain at least a portion of the elongate body in the urethra.

In an exemplary embodiment, the urological medical device includes a sensor adapted to sense fluid.

In an exemplary embodiment, the urological medical device includes a plurality of pressure sensors, spaced apart along a length of the elongate body, and a processing unit. The processing unit is in communication with the plurality of pressure sensors and is configured to synchronously read a pressure from each of the plurality of pressure sensors.

A urological medical device according to an exemplary embodiment of the present invention includes an elongate body adapted to be placed in a patient's urethra. The elongate body includes a plurality of pressure sensors spaced apart along a length of the elongate body. At least a portion of the elongate body is radially expandable. The urological medical device further includes a processing unit in communication with the plurality of pressure sensors configured to synchronously read the pressure from each of the plurality pressure sensors when the elongate body is placed in the urethra.

In an exemplary embodiment, the processing unit is further configured to compute a compliance of the urethra based on pressure and/or spatial orientation readings from the pressure sensors.

In an exemplary embodiment, the elongate body includes a lumen and is adapted to stretch circumferentially upon application of a pressure in the lumen, e.g., of greater than approximately 5 cm H₂0.

In an exemplary embodiment, the elongate body is a balloon catheter.

In an exemplary embodiment, the urological medical device further includes stylets of varying diameter, wherein each of the predetermined circumferences of the elongate body are achieved by inserting the stylets one at a time through a lumen extending along a length of the elongate body. The elongate body is sufficiently flexible to a stretch upon insertion of the stylet into the lumen, each stylet expanding the elongate body to one of the predetermined circumferences.

In an exemplary embodiment, the pressure sensors are configured to measure a pressure of a fluid or gas within the elongate body.

In an exemplary embodiment, the pressure sensors are configured to measure a pressure within the urethra outside the elongate body.

An exemplary method of the present invention for analyzing urethral properties, includes inserting a highly flexible elongate body into a patient's urethra and tracking movement of discrete points along a length of the body over a predetermined time period.

In an exemplary embodiment, the discrete points are tracked synchronously.

In an exemplary embodiment, the method further includes determining which of the discrete points moves the most during the predetermined time period.

In an exemplary embodiment, the patient at least one of coughs, sneezes, laughs, and squeezes his or her abdomen muscles during the predetermined time period causing involuntary leakage of fluid through the urethra.

In an exemplary embodiment, the method further includes displaying movement of the elongate body represented by the discrete points in real time.

In an exemplary embodiment, the method further includes displaying a flow of urine at least one of through or around the elongate body.

An exemplary method of the present invention for analyzing urethral properties, includes inserting an elongate body into a patient's urethra and using the elongate body to measure a resistance of the urethra to radial expansion synchronously at a plurality of points along a length of the urethra.

In an exemplary embodiment, the method further includes detecting urine flow at least one of through and around the elongate body.

In an exemplary embodiment, the resistance of the urethra to radial expansion is measured while urine is passing through the urethra.

In an exemplary embodiment, the resistance of the urethra to radial expansion is measured by (i) tracking a position of each of the plurality of points, and (ii) measuring the pressure in the urethra at each of the positions.

In an exemplary embodiment, the elongate body is expanded by one of (i) passing a stylet into a lumen defined by the elongate body, the stylet having a diameter larger than that of the lumen, and (ii) forcing gas or fluid into the lumen at a pressure sufficient to expand at least a portion of the elongate body.

An exemplary method of the present invention for treating urinary incontinence, includes the steps of: at least one of (i) making a periurethral injection, and (ii) placing a support element adjacent, e.g., beneath or alongside, the urethra, wherein a location of the periurethral injection and the support element along a length of the urethra is chosen based on at least one of (i) a compliance of the urethra during incontinence, and (ii) a degree to which the urethra changes shape during incontinence. As used herein the term support element includes any device known in the art to suspend or provide support or positional guidance to the urethra, such as, but not limited to, a suburethral sling and suspension sutures.

According to an exemplary embodiment, the support element is placed adjacent a most compliant portion of the urethra and/or the periurethral injection is made at the most compliant portion of the urethra.

According to an exemplary embodiment, the support element is placed adjacent a portion of the urethra that undergoes the largest degree of bending during incontinence as compared to other portions of the urethra and/or the periurethral injection is made at the portion of the urethra that undergoes the largest degree of bending during incontinence.

According to an exemplary embodiment, one or more periurethral injections are made into the urethra. The elongate body is inserted into the urethra and used to determine the effect, if any, the injection had on continence. The elongate body may be maintained inside the urethra during injection or placed in the urethra after each injection. The extent to which the injection changes the properties of the urethra determines whether further injections are necessary to sufficiently reduce incontinence.

An exemplary method of the present invention for optimally placing a support element to treat urinary incontinence, includes the steps of: placing the support element adjacent, e.g., beneath or alongside, a portion of the urethra such that the support element exerts a pressure against the portion, the pressure chosen based on a compliance of at least a portion of the urethra.

In an exemplary embodiment, a first pressure is applied when the portion of the urethra has a compliance below a predetermined compliance and a second pressure is applied when the portion of the urethra has a compliance above the predetermined compliance, the first pressure being higher than the second pressure.

An exemplary method of the present invention for analyzing urethral properties, includes the steps of: (a) inserting an elongate body into a patient's urethra such that one end extends into the patient's bladder, at least one pressure sensor on the elongate body is inside the urethra, at least one pressure sensor on the elongate body is located external to the patient, and at least one pressure sensor on the elongate body is inside a bladder of the patient; and (b) using the pressure sensors to locate at least one of (i) the urethral meatus, and (ii) a proximal extent of the patient's sphincter.

In an exemplary embodiment, the locations of the urethral meatus and the proximal extent of the sphincter are determined by looking for a predetermined difference in pressure between adjacent pressure sensors.

In an exemplary embodiment, the urological medical device includes a control unit in communication with the pressure sensors and one or more sensors on the elongate body adapted to sense fluid. The elongate body is placed in the urethra and the control unit is adapted to at least one of (i) compute a vesical leak point pressure (VLPP), (ii) determine a point in the urethra where urethral pressure (Pure) becomes greater than vesical pressure (Pves), (iii) compute bladder neck descent during incontinence, (iv) compute a resting urethral angle, (v) compute a urethral angle during incontinence, (vi) compute a compliance of the urethra during incontinence, (vii) track movement of various points along a length of the elongate body during incontinence and determine which point moves the most, (viii) determine an anatomic location of the urethral meatus, and (ix) determine an anatomic location of a proximal extent of the sphincter.

Reference throughout this specification to “an embodiment” or “one embodiment” or “an exemplary embodiment” means that a particular feature, structure, or characteristic described in connection with the embodiment is included in at least one embodiment of the present invention. Thus, the appearances of these phrases in various places throughout this specification are not necessarily all referring to the same embodiment. Furthermore, the particular features, structures, or characteristics may be combined in any suitable manner in one or more embodiments.

An example embodiment of the present invention is described in more detail below with reference to the appended Figures. The foregoing description and examples have been set forth as mere illustrations and are not intended to be limiting. Each of the disclosed aspects and embodiments may be considered individually or in combination with other aspects, embodiments, and variations thereof. The steps of the methods described herein are not confined to any particular order of performance.

### BRIEF DESCRIPTION OF THE SEVERAL VIEWS OF THE DRAWINGS

FIG. 1A is a schematic illustration of an exemplary embodiment of the present invention.

FIG. 1B is an enlarged view of the proximal end of the elongate body of FIG. 1A.

FIG. 1C is the elongate body proximal end of FIG. 1B forced into a J-shaped configuration.

FIG. 1D is a schematic illustration of an exemplary embodiment of the present invention.

FIG. 2A is a schematic illustration of another exemplary embodiment of the present invention.

FIG. 2B is a schematic illustration of the exemplary embodiment of FIG. 2A with the retention element closed.

### DETAILED DESCRIPTION OF THE INVENTION

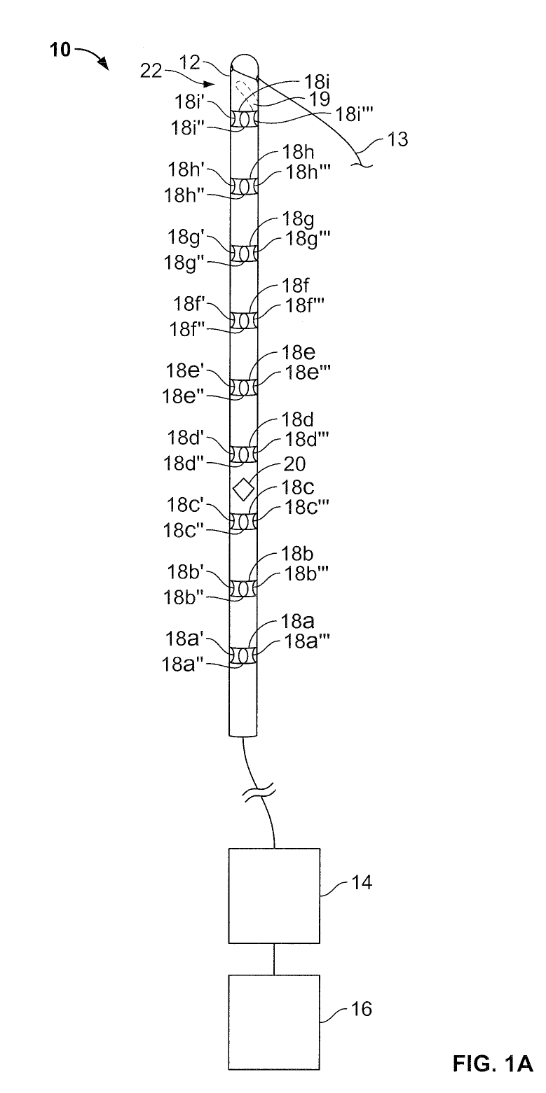

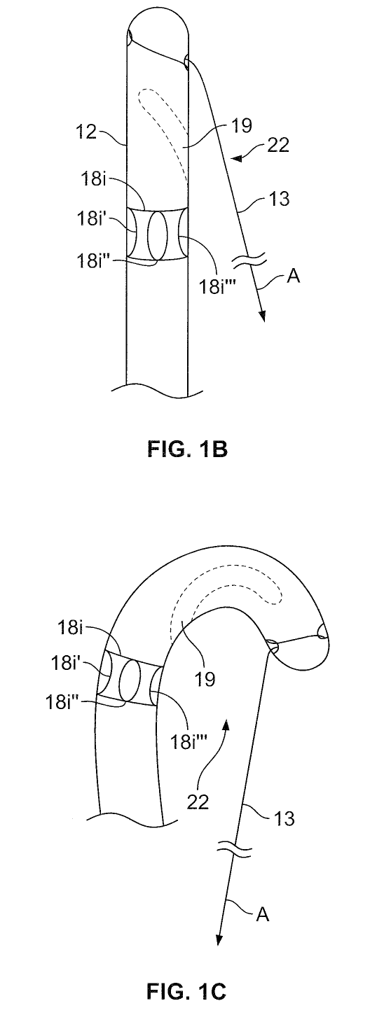

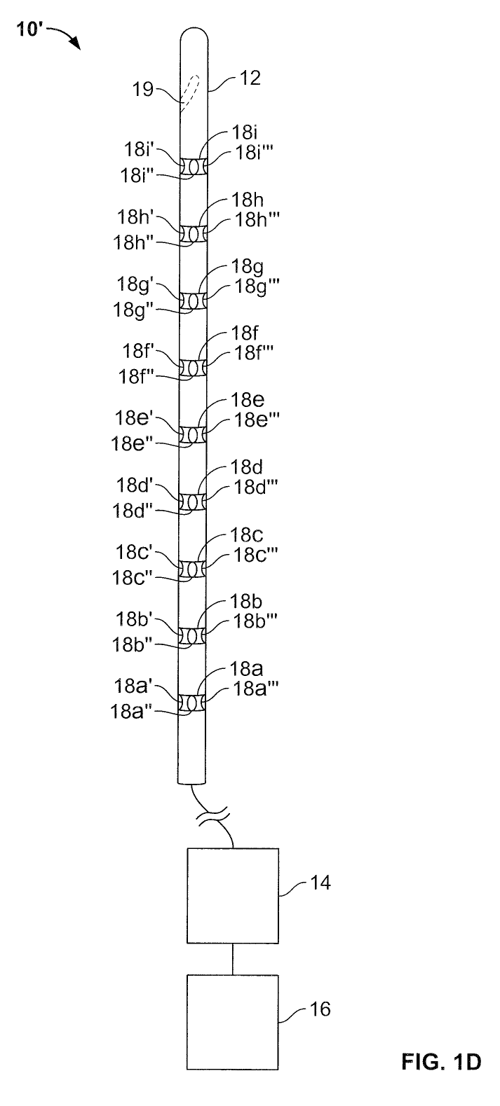

FIG. 1 is a perspective view of an urological medical device according to an exemplary embodiment of the present invention. The device includes an elongate body 10 adapted to be inserted into the urethra of a patient, proximal end 12 first. The elongate body 10 communicates with a control unit 14, which in turn communicates with a monitor display 16, which can be separate from or integrated with the control unit 14. As illustrated the elongate body 10, control unit 14, and display 16 have a hardwire and/or physical connection but they may also communicate wirelessly.

The elongate body 10 can be inserted into a patient, for example, using a stiff insertion rod. A proximal end of the rod is disposed in a lumen 19 (shown in dashed lines) and advanced into the urethra with the elongate body 10 until the proximal end 12 lies in the bladder of the patient. Other methods for insertion may be used as well, including the use of an insertion sheath. The elongate body 10 may also be inserted by manually advancing it into the urethra and bladder without the assistance of an insertion rod or sheath.

The elongate body 10 is highly flexible, i.e., flexible enough to take on the shape of the urethra in which it is disposed. The elongate body may have properties similar to or even more flexible than that of a highly flexible plastic worm fishing lure. Such lures are so flexible that when lengths as short as three or four centimeters are held vertically from below they collapse or buckle under their own weight. Further, they are capable of being bent 180 degrees (such that each of the two ends point in the same direction and are parallel) without permanently deforming.

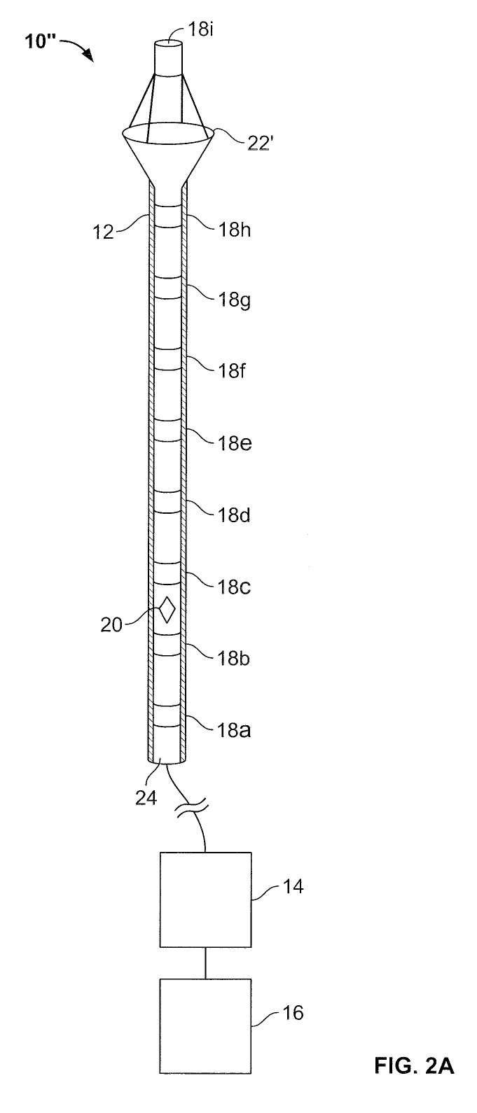

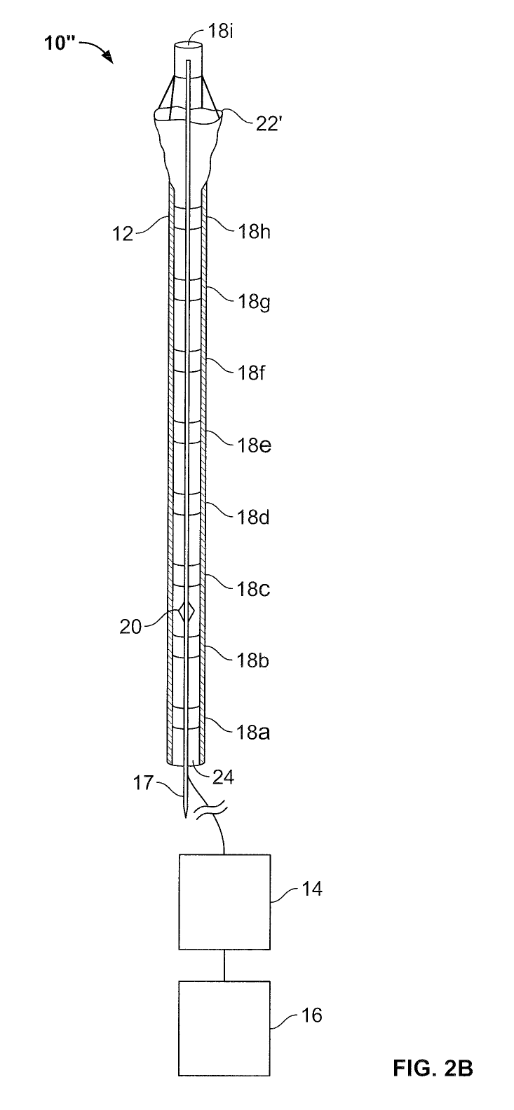

This degree of flexibility can be accomplished in the context of a urological device sized to be placed in the urethra by using a very soft material, e.g., a durometer less than approximately 10 on the Shore A scale, as used in the embodiment of FIG. 1, and/or by configuring the body to facilitate bending, e.g., by using a hollow body with very thin walls consistent with the embodiment of FIG. 2A. For example, a super thin condom-like tube or a tube with kinks or weakened circumferential sections may be used.

Typical urethral catheters are much stiffer. For example, a three or four centimeter length of a typical prior art urethral tubing or catheter, when held vertically from below, will hardly bend or sway from its vertical longitudinal axis, let alone buckle or collapse under its own weight. This is true even if for a length twice as long, i.e., six or seven centimeters long. Urethral catheters are typically designed with a certain level of rigidity to facilitate insertion into the urethra and/or to assure that a lumen defined by the catheter remains open. Stiffer catheters are less prone to buckling and kinking while being advanced into the urethra and during movement of the patient.

The elongate body 10 may be made in conventional fashion by molding, e.g., injection molding, a soft yieldable flexible synthetic rubber or plastic composition as is known in the art. In an exemplary embodiment, a silicone elastomer such as manufactured by Dow Corning under the designation Q7-4840 may be used, but other elastomers of silicone rubber, polyurethane, latex, or any of a variety of other similar materials may also be used.

In order to achieve the low durometer for elongate body 10 a variety of materials may be used. For purposes of example only and not limitation, a mixture of materials consisting primarily or mostly of the following polymeric materials may be used: Dioctyl Phthalate (DOP), Polyvinyl Chloride (PVC), and an elastomeric polymer emulsion (EPE). In an exemplary embodiment, the combination should be approximately 51 to 66.6% of DOP, approximately 11-22.4% of PVC, approximately 5-28% of EPE and approximately 2-6% of other materials. The use of additional PVC has the effect of hardening the resulting material. Conversely, to decrease durometer hardness the PVC component should be decreased.

The elongate body 10 includes a plurality of spaced apart pressure sensor rings 18a, 18b, 18c, 18d, 18e, 18f, 18g, 18h, and 18i connected to the elongate body 10 along its length. A larger or smaller number of pressure sensors may be used as well. When the elongate body 10 is inserted into a patient, proximal pressure sensors, e.g., sensors 18h and 18i, are placed in the bladder, at least one distal pressure sensor, e.g., sensor 18a, remains outside the urethra distal the urethral meatus, and at least one intermediate pressure sensor, e.g., sensors 18b, 18c, 18d, 18e, 18f, and 18g, are positioned inside the urethra.

Each pressure sensor ring 18a-i includes four individual pressure sensors spaced ninety degrees apart (only three of which are visible in FIG. 1). A larger or smaller number of sensors may be used and their circumferential placements may also vary. Pressure sensor ring 18a includes pressure sensors 18a′, 18a′, 18a′″, 18a″″ (not shown). The pressure sensors on the remaining rings 18b-18i are labeled using the same methodology, each ring bearing its own letter, etc. For clarity, the individual pressure sensors are not shown in FIGS. 2A and 2B.

The four pressure measurements at each ring may be averaged to provide a single pressure reading at each ring. The control unit 10 may also screen out certain pressure measurements before calculating an average to the extent the measurement clearly represents an error, e.g., is outside a predetermined range.

The pressure sensors may comprise piezoelectric pressure transducers, which, for example, communicate wirelessly with the control unit 14 or are hardwired via one or more wires running along a length of the elongate body 10. The pressure at one or more of the pressure sensors may also be communicated to the control unit 14 through a diaphragm and via a dedicated fluid column disposed in a lumen in the elongate body 10.

Tracking members may be connected along a length of the elongate body 10 so as to allow tracking of the shape and positioning of the elongate body 10 while inside the patient. The position and movement of the tracking members are tracked by the control unit 14 relative to a fixed marker in the control unit 14. Alternatively, a separate locator unit in communication with the control unit 14, e.g., placed adjacent to the patient, may be used to track the movement of the tracking members. As illustrated in FIG. 1, the pressure sensors in rings 18a-18i serve as the tracking members but separate tracking members may also be used.

The tracking members may, for example, be ultrasonic receivers, such as ultrasonic piezoelectric transducers, and the locator unit may include an echoscope and a transducer probe used to track the transducers. The elongate body 10 may be made from a material that is ultrasonically transparent. See, for example, U.S. Pat. No. 4,697,595, herein incorporated in its entirety by reference thereto. Other types of tracking elements known in the art may also be used. The elongate body 10 may also be tracked using X-ray or fluoroscopy. See, for example, U.S. Pat. Nos. 4,697,595, 6,904,308, and 6,958,034, herein incorporated in their entireties by reference thereto.

Given its highly flexible nature, the elongate body 10 takes on the shape of the urethra in which it is disposed. As the urethra moves, the elongate body 10 moves with it providing minimal resistance. Movement of the urethra, therefore, may be tracked via tracking of the tracking elements on the elongate body 10.

An outline or representation of the urethra may be generated by the control unit 14 and displayed on the monitor display 16. The representation may be formed by displaying one point or node for each of the tracking members, e.g., spaced 5 to 10 mm apart. The position of the these nodes is dictated by their position relative to the fixed marker point and/or their relative positioning as determined by the control unit 14. Each node is connected to its adjacent node by a straight or curved line segment so as to create a representation or image of the urethra. In this manner, a clinician can visualize and track movement of the urethra in particular during periods of incontinence.

Incontinence may be detected visually or via fluid sensors 20. Fluid sensors 20 may include an electrical conductor including two metallic rings. When urine flows over the rings an electrical connection is made or a resistance is reduced, which triggers an alarm. See, for example, U.S. Pat. No. 6,056,699, herein incorporated in its entirety by reference thereto. The control unit 14 may mark the data points of pressure and tracking member locations at the time of leakage so that synchronous events can be displayed. An audible signal may also be emitted when fluid sensors 20 detect flow. As shown, fluid sensors 20 are located adjacent a distal end of the elongate body but they can also be located at other portions or along an entire length of the elongate body 10. Multiple fluid sensors would allow for a graphic representation on the monitor display 16 of urine flowing through the urethra.

The control unit 14 may be configured to synchronously track the position of the tracking elements and/or to synchronously take pressure readings from the pressure sensors 18a-18icontinuously and/or at predetermined intervals and/or during periods of incontinence, as detected, e.g., by the fluid sensors 20. The measurement sequence of the control unit 14 may also be triggered manually by a clinician.

Synchronous measurement, as used herein, means at the same time or very close in time and is intended to provide a snapshot of the urethra. Synchronous pressure readings and tracking readings during incontinence provide the clinician with a true physiologic understanding of the anatomy during incontinence, which facilitates both urological function assessment and optimal placement of a periurethral injection and/or a support device, such as a suburethral sling and suspension sutures, during surgery.

A diameter of the elongate body 10 for use in an adult patient can be approximately 2.3 mm. The device may also be downsized for use smaller populations as well as children. The typical urethra at rest is collapsed and during voiding (and incontinence) can expand to approximately 10 mm.

In an exemplary embodiment, a retention element 22 is connected to proximal end 12 of the elongate body 10, which may be used to fix proximal end 12 in the bladder. The retention element 22 may include proximal end 12 connected to a line 13. Alternatively, an umbrella-like retention element similar to that illustrated in connection with the embodiment of FIG. 2A may be used.

For clarity, the proximal end 12 of elongate body is shown independently in FIG. 1B. Once placed in the bladder, line 13 is pulled distally in the direction of arrow A causing the proximal end 12 to take on a J-shaped configuration, as shown in FIG. 1C, allowing it to sit at the bladder neck without sliding into the urethra while the patient applies abdominal pressure. Line 13 is shown connected on one end to the elongate body 10 but may also pass under ring 18i or a collar around the elongate body 10 (not shown) to keep the line close to the side of the elongate body 10. Further, line 13 is shown wrapped around proximal end 12 but may be connected to the elongate body 10 in other ways known in the art, e.g., using an adhesive.

In another exemplary embodiment, as illustrated in FIG. 1D, the elongate body 10′ is designed long enough to obviate the need for a retention element. For example, the elongate body 10′ may be designed to assure that at least, e.g., a ten centimeter length remains in the bladder at all times. The elongate body 10′ will likely move during application of abdominal pressure by the patient, perhaps as much as eight centimeters, but given its long length at least a portion of the elongate body 10′ will remain in the bladder, thus obviating the need for a retainer element.

The control unit 14 may use the pressure sensor rings 18A-18i or tracking members to keep track of the position of the elongate body 10, 10′, 10″ relative to points in the body, e.g., the urethral meatus and the proximal extent of the sphincter, notwithstanding movement caused by coughing, laughing, abdominal pressure, etc. The urethral meatus location will be assigned along the inserted elongate body 10, 10′, 10″ by selecting, e.g., the location of the most proximal pressure sensor ring that is closest to the point that urethral pressure falls to atmospheric pressure. This may be done, for example, by looking for two adjacent pressure rings that read different pressures consistent with one ring being inside the urethra and the other outside the urethra. The urethral meatus position may be assigned to either one of the rings or a position between the rings.

Similarly, the elongate body 10′ is first positioned such that retention element 22 fits snugly against the bladder neck, marking the anatomic location of the proximal sphincter, e.g., as the position of ring 18h. The control unit 14 compares the readings of all adjacent pressure sensors and looks for the position at which Pure first becomes a predetermined amount greater than Pves and marks this as the proximal extent of the sphincter at rest. The position at which Pves is equal to or greater than Pure in the distal urethra is marked as the distal extent of the sphincter at rest. When the patient is asked to cough or strain, the control unit 14 compares the readings of all adjacent pressure sensor rings and looks for a difference above a predetermined amount, e.g., which is consistent with the pressure difference between the urethra and the bladder. To the extent this pressure difference shifts, for example, from between pressure rings 18g and 18h to more distal pressure rings 18f and 18e, e.g., when the patient coughs, the control unit 14 will reassign a location of the proximal extent of the sphincter during cough or strain, e.g., to a point along the elongate body 10 at ring 18f or 18e. Movement is not an issue for the embodiment of FIGS. 1A and 2A so that the elongate body 10 and 10″ may be positioned such that only single pressure sensor unit, e.g., pressure sensor ring 18i lies in the bladder.

The position of the urethral meatus and the proximal extent of the anatomic sphincter can also be used in generating the display of the urethra on the monitor display 16 and in calculating a length of the urethra.

FIGS. 2A and 2B illustrate an exemplary embodiment of the present invention including a thin walled highly flexible elongate body 10″ resembling a condom but open on both sides. The elongate body 10″ defines a lumen 24 extending a full length of the elongate body 10″. The elongate body 10″ may be made, e.g., from a plastic or elastomer, and may have a very thin wall thickness, e.g., in a range of from about 1 to about 25 mils. In an exemplary embodiment, the wall thickness may be in a range of from about 2 to about 10 mils. In another exemplary embodiment, the wall thickness may range from about 2 to about 6 mils. Lumen 24 may have an internal size of 1 French expandable up to 30 French.

The polymeric film material used to fabricate the elongate body 10″ may be used, e.g., in the form of a blown film, extruded sheet, solvent cast film or other suitable web stock formed of the polymeric material.

Among polymeric film materials useful in the broad practice of the present invention to form the elongate body 10″, illustrative materials include: polyurethane; styrene-isoprene-styrene/styrene-butadiene-styrene compositions, such as Kraton® polymers (commercially available from Shell Chemical Company, Houston, Tex.); polyvinylchloride (PVC) that has been plasticized to the desired flexibility; urethane/PVC blends; urethane that has been plasticized to the desired flexibility; Covale™ polymer (commercially available from Dow Chemical Company, Midland, Mich.); polyester elastomers such as Hytrel® (commercially available from E.I. DuPont de Nemours & Company, Wilmington, Del.); polyamide elastomers such as Pebax® (commercially available from Atochem); olefinic polymers (polypropylene, polyethylene, etc.); and metallocene polymers.

A stylet 17 may be disposed within or placed alongside the elongate body 10″ and used to advance the device into the urethra such that the retention element 22′ lies in the bladder. Movement of the stylet 17 relative to the elongate body 10″ stretches the retention element 22′ from its resting position as seen in FIG. 2A (resembling an umbrella) to its insertion position as seen in FIG. 2B (resembling a closed umbrella). The stylet 17 may be removed upon insertion of the device in the patient (it is not shown in FIG. 2A) and replaced upon removal of the device. When the stylet 17 is removed, the retention element 22″ assumes its resting position preventing withdrawal of the elongate body 10″ through the urethra. Other types of retention elements known in the art may be used as well, e.g., the retention element on the percutaneous suprapubic catheter sold by Cook Urological.

Similar to the embodiment of FIG. 1, the elongate body 10″ of FIG. 2 includes pressure sensor rings 18a-18i or tracking members as well as a fluid sensor 20. In this embodiment, however, during periods of incontinence urine travels through the elongate body lumen 24 as opposed to around the elongate body 10 (FIG. 1). The retention element 22′ functions as a funnel directing the urine into the lumen 24. As illustrated, the pressure sensor rings 18a-18i are on an outside surface of the elongate body 10″ and the fluid sensor 20 is on an inside surface of the elongate body 10″. However, the pressure sensor rings 18a-18i may also be inside the elongate body 10″.

During incontinence, urine flows through the retention element 22′ into the lumen expanding the elongate body 10″ and the urethra circumferentially. The elongate body 10″ is circumferentially expandable under pressures consistent with that which the urethra is exposed during periods of incontinence, e.g., 5 to 200 cm H₂0. For example, the elongate body 10″ may be expandable from an initial diameter of _{—}1 mm to an expanded diameter of 10 mm. The degree to which the elongate body 10″ circumferentially expands will depend in part on the compliance of the urethra in which the elongate body 10″ is disposed.

The control unit 14 is configured to track the position of the pressure sensors and/or tracking members and calculate an expanded circumference at each pressure sensor/tracking member ring along the length of the elongate body 10″. For example, as urine flows through the elongate body 10″ pressure sensors 18d, 18d′, 18d″ and 18d′″ move radially away from each other. At predetermined time intervals, the control unit 14 measures the pressure at each of these pressure sensors and stores their relative location in an integrated or external memory unit. The control unit 14 performs measurements at each of the other pressure sensors, e.g., synchronously with pressure sensors 18d, 18d′, 18d′″, and 18d′″. The control unit 14 then uses the relative locations of the pressure sensors in each ring to arrive at an expanded circumference and uses this information to calculate a compliance at each corresponding point along the urethra. The compliance of the urethra at each pressure sensor location can be calculated by dividing the change in circumference at that location by the change in pressure at that location over a predetermined period of time. The circumference of the urethra at various points, can also be used to determine the urethral shape, which as indicated above, may be displayed, e.g., during urine flow, using display 16.

The embodiment of FIG. 1 may be provided with a pressurized fluid source and a communicating lumen down the length of the elongate body 10 (terminating before its proximal end) allowing it also to be used to measure compliance of the urethra. Fluid from the fluid source may flow into the elongate body 10 while it is in the urethra causing it and the urethra to circumferentially expand to a plurality of predetermined diameters. At each predetermined diameter the control unit 14 may read the pressure at each of the pressure sensor rings 18a-18i and calculate a compliance of the urethra, as further detailed above. Alternatively, rather than fill the lumen with fluid or gas to achieve the expanded circumferences, stylets of varying predetermined sizes may be inserted into the lumen one at a time so as to expand the elongate body 10 and the urethra to the predetermined circumferences.

The controller 14 may calculate a number of useful patient parameters based on readings from the fluid and pressure sensors and/or tracking members and may communicate this information to the clinician, e.g., via the display 16 or audibly using a speaker. For example, the controller 14 may communicate the vesical leak point pressure (VLPP), which is the lowest pressure in the bladder when the fluid sensor 20 first detects urine. VLPP is a measure of sphincteric strength and a prognostic feature for successful treatment. If VLPP is low (especially if there is no urethral mobility) suburethral slings or suspension sutures are more likely to be successful than other kinds of surgery.

The controller 14 may also communicate the pressure in the patient's urethra when resting, i.e., resting Pure, which is another measure of sphincter strength.

The controller 14 may communicate urethral transmission pressures, which is the ratio of Pure, the pressure in the urethra, to Pves, the pressure in the bladder, obtained during coughing or straining. Theoretically, as long as the ratio is 100% or greater, continence is preserved. Any of the pressure sensors on the elongate body 10, 10′, 10″ in the bladder can be used to glean Pves and any of the pressure sensors on the elongate body 10, 10′, 10″ in the urethra can used to glean Pure. The controller 14 may also communicate changes in Pure at points along the urethra during increases in abdominal pressure, i.e., Pabd, which is one factor used to calculate transmission pressure. The controller 14 may receive input from the clinician to mark the point in time when the patient bears down or coughs or otherwise voluntarily contracts the sphincter to increase Pabd.

The controller 14 may also communicate the active and passive anatomic points in the urethra where Pure becomes greater than Pves during increased abdominal pressure. This point represents the functional location of the proximal extent of the sphincter.

As indicated above, a model of the urethra is generated by the controller 14 after determining the location of the urethral meatus, the anatomic position of the bladder neck, and the distal extent of the sphincter. Based on this model, the controller 14 may also communicate the active and passive functional urethral length, i.e., the length from the anatomic bladder neck (determined, e.g., by pulling the retention element 22, 22′ against it) to the point in the urethra where Pure>Pves. This, coupled with the length of the urethra defines the anatomic location of the entire sphincter. The controller 14 may update the functional urethral length as Pves changes, e.g., continuously or on a predetermined basis or when Pves changes a predetermined amount.

The controller 14 may also communicate the anatomic urethral length, i.e., the distance along the urethra between the urethral meatus and the bladder neck. This is one of the factors that may be used to calculate the anatomic position of the sphincter and a number of functional points along the urethra. The distal end of the urethra is one fixed point that is used for calculation of anatomic and functional points that determine specific goals of surgery, i.e., where to place a suburethral sling or suspension sutures or where to make a periurethral injection.

The controller 14 may also communicate the amount of bladder neck descent, e.g., measured in relation to the urethral meatus. In certain circumstances, the lowest point of bladder neck descent may be the most distal point that a suburethral sling or suspension sutures should be placed, i.e., the furthest point along the urethra away from the bladder.

The controller 14 may also communicate resting urethral angle, which is a point of reference for urethral mobility. Urethral angle may be calculated by drawing a straight line between the urethral meatus and the proximal extent of the sphincter and measuring the angle this line forms relative to the horizontal. The controller 14 may also communicate the change in the urethral angle during increases in Pabd. This change in urethral angle is representative of urethral mobility, which is one of the prognostic features for successful treatment. The greater the mobility, the more likely that any kind of anti-incontinence surgery will be effective and the less likely that periurethral injections will be effective.

The controller 14 may also communicate the relationship between the change in urethral angle and the change in Pves, e.g., using a plot. This relationship is a measure of the force necessary to move the bladder neck and urethra and in a sense a measure of pelvic floor strength.

The controller 14 may also communicate the relationship between the change in urethral angle and the change in Pure, e.g., using a plot. This relationship is a measure of sphincter strength. This parameter is useful for choosing between treatment options. A periurethral injection may suffice for a sedentary patient with a high VLPP and little mobility. In contrast, a very active patient with the same VLPP but high mobility may require a suburethral sling and/or suspension sutures.

The controller 14 may also communicate the change in urethral circumference during increases in Pabd, which is another measure of sphincter strength.

The controller 14 may also communicate the urethral compliance at different points along the urethra. While urethral compliance is not presently part of the lexicon of incontinence, it is known that patients with a “pipe stem urethra,” i.e., an extremely stiff urethra, have a very poor prognosis with respect to the surgical correction of incontinence.

A clinician may use all of the information communicated by the controller to assess urological function and to optimally place a periurethral injection and/or a support element, such as a suburethral sling or suspension suture. To date, surgeons typically place the suburethral sling and suspension sutures somewhere towards a middle of the urethra without any tension at all. The inventors of the present invention believe that the position of the support element along the length of the urethra and the pressure at which the support element contacts the urethra are critical to the success of the surgery. Rather than simply placing the support element in the middle of the urethra, the support element should optimally be placed at a portion of the urethra demonstrating the largest change in position during increase in Pabd or at a region demonstrating the highest compliance. Further, the lower the compliance of this portion, the more pressure should be applied by the support element against the urethra. The inventor believes that additional pressure is necessary for low compliant urethras so as to assure that the urethra walls are compressed against the support element during urethra movement, which is necessary to maintain continence.

The controller 14 may be programmed to analyze all of the measured and calculated parameters and provide a suggestion as to support element positioning and pressure. For example, a suburethral sling or suspension suture may actually be displayed on the display 16 adjacent the model of the urethra and/or coordinates provided, e.g., relative to the urethral meatus. Real time visualization of the urethra movement during incontinence will also facilitate the decision as to the placement of the suburethral sling and suspension suture.

In an exemplary embodiment, while the elongate body 10, 10′, 10″ is placed in the urethra, one or more periurethral injections are made into the urethra. After each injection, the elongate body 10, 10′, 10″ is used to gauge the effect of the injection on the urethra.

Those skilled in the art can appreciate from the foregoing description that the present invention can be implemented in a variety of forms. Therefore, while the embodiments of this invention have been described in connection with particular examples thereof, the true scope of the embodiments of the invention should not be so limited since other modifications and variations will become apparent to the skilled practitioner upon a study of the drawings and specification. Such modifications and variations are considered to be within the purview and scope of the appended claims and their equivalents.

What is claimed is:

1. A urological medical device comprising an elongate body adapted to be inserted into the urethra of a patient, the body is highly flexible along its length at normal body temperatures.

2. The urological medical device of claim 1, wherein the elongate body is made entirely from an elastomeric material.

3. The urological medical device of claim 1, wherein the elongate body is made from an elastomeric material having a durometer of less than 30 on the Shore A scale at normal body temperatures.

4. The urological medical device of claim 1, wherein the elongate body is made from an elastomeric material having a durometer of less than 20 on the Shore A scale at normal body temperatures.

5. The urological medical device of claim 1, wherein the elongate body is made from an elastomeric material having a durometer of less than 10 on the Shore A scale at normal body temperatures.

6. The urological medical device of claim 1, wherein the elongate body is made from an elastomeric material having a durometer of less than 5 on the Shore A scale at normal body temperatures.

7. The urological medical device of claim 1, wherein the elongate body includes a lumen extending longitudinally along an entire length of the elongate member, the body having a radial wall thickness of less than or equal to approximately 25 mils.

8. The urological medical device of claim 1, wherein the elongate body includes a lumen and is adapted to stretch circumferentially upon application of a pressure in the lumen greater than approximately 5 cm H₂0.

9. The urological medical device of claim 1, further comprising a plurality of tracking members fixed to the elongate body, and a locating device configured to track the position of the tracking members.

10. The urological medical device of claim 1, further comprising one or more pressure sensors connected to the elongate body.

11. The urological medical device of claim 9, wherein the tracking members also function as pressure sensors.

12. The urological medical device of claim 1, further comprising one or more groups of two or more pressure sensors, each of the two or more pressure sensors connected to the elongate body at the same point along a length of the elongate body but spaced apart from each other circumferentially.

13. The urological medical device of claim 12, wherein each of the one or more groups are spaced apart from each other along the length of the elongate body.

14. The urological medical device of claim 1, further comprising a retaining element on one end of the elongate body and configured to removably retain at least a portion of the elongate body in the urethra.

15. The urological medical device of claim 1, wherein the elongate body includes a proximal tip having a lumen adapted to receive a proximal end of an insertion element used to insert the elongate body into the urethra of a patient.

16. The urological medical device of claim 1, wherein the elongate body has a length greater than 30 cm and does not have a retention means used to removably retain at least a portion of the elongate body in the urethra.

17. The urological medical device of claim 1, further comprising a sensor adapted to sense fluid.

18. The urological medical device of claim 1, further comprising a retention element at a proximal end of the elongate body adapted to removably retain the elongate body in the urethra.

19. The urological medical device of claim 1, further comprising a plurality of pressure sensors spaced apart along a length of the elongate body, and a processing unit in communication with the plurality of pressure sensors configured to synchronously read a pressure from each of the plurality of pressure sensors.

20. A urological medical device comprising:
an elongate body adapted to be placed in a patient's urethra and including a plurality of pressure sensors spaced apart along a length of the elongate body, at least a portion of the elongate body radially expandable; and
a processing unit in communication with the plurality of pressure sensors configured to synchronously read the pressure from each of the plurality pressure sensors when the elongate body is placed in the urethra.

21. The urological device of claim 20, wherein the processing unit is further configured to compute a compliance of the urethra based on the pressure readings.

22. The urological device of claim 20, wherein the elongate body includes a lumen and is adapted to stretch circumferentially upon application of a pressure in the lumen of greater than 5 cm H₂0.

23. The urological device of claim 20, wherein the pressure sensors are configured to measure a pressure of a fluid or gas within the elongate body.

24. The urological device of claim 20, wherein the pressure sensors are configured to measure a pressure within the urethra outside the elongate body.

25. A method for analyzing urethral properties, comprising the steps of:
inserting a highly flexible elongate body into a patient's urethra; and
tracking movement of discrete points along a length of the body over a predetermined time period.

26. The method of claim 25, wherein the discrete points are tracked synchronously.

27. The method of claim 25, further comprising determining which of the discrete points moves the most during the predetermined time period.

28. The method of claim 25, wherein the patient at least one of cough's, sneezes, laughs, and squeezes his or her abdomen muscles during the predetermined time period causing involuntary leakage of fluid through the urethra.

29. The method of claim 25, further comprising the step of displaying movement of the elongate body represented by the discrete points in real time.

30. The method of claim 25, further comprising the step of displaying a flow of urine at least one of through or around the elongate body.

31. A method for analyzing urethral properties, comprising the steps of:
inserting an elongate body into a patient's urethra;
using the elongate body to measure a resistance of the urethra to radial expansion synchronously at a plurality of points along a length of the urethra.

32. The method of claim 31, further comprising the step of detecting urine flow at least one of through and around the elongate body.

33. The method of claim 31, wherein the resistance of the urethra to radial expansion is measured while urine is passing through the urethra.

34. The method of claim 31, wherein the resistance of the urethra to radial expansion is measured by (i) tracking a position of each of the plurality of points, and (ii) measuring the pressure in the urethra at each of the positions.

35. A method for treating urinary incontinence, comprising the steps of:
at least one of (i) placing a support element adjacent the urethra and (ii) making a periurethral injection, wherein a location of the support element and the periurethral injection along a length of the urethra is chosen based on at least one of (i) a compliance of the urethra during incontinence, and (ii) a degree to which the urethra changes shape during incontinence.

36. The method of claim 35, wherein at least one of (i) the periurethral injection is made into most compliant portion of the urethra, and (ii)the support element is placed adjacent most compliant portion of the urethra.

37. The method of claim 35, wherein at least one of (i) the periurethral injection is made into a portion of the urethra that undergoes the largest degree of bending during incontinence as compared to other portions of the urethra, and (ii) the support element is placed adjacent the portion of the urethra.

38. The method of claim 35, wherein the support element is one of a suburethral sling and a suspension suture.

39. A method for optimally placing a support element to treat urinary incontinence, comprising the steps of:
placing a support element adjacent a portion of the urethra such that the support element exerts a pressure against the portion, the pressure chosen based on a compliance of at least a portion of the urethra.

40. The method of claim 39, wherein a first pressure is applied when the portion of the urethra has a compliance below a predetermined compliance and a second pressure is applied when the portion of the urethra has a compliance above the predetermined compliance, the first pressure being higher than the second pressure.

41. The method of claim 39, wherein the support element is one of a suburethral sling and a suspension suture.

40. A method for analyzing urethral properties, comprising the steps of:
inserting an elongate body into a patient's urethra such that one end extends into the patient's bladder, at least one pressure sensor on the elongate body is inside the urethra, at least one pressure sensor on the elongate body is located external to the patient, and at least one pressure sensor on the elongate body is inside a bladder of the patient;
using the pressure sensors to locate at least one of (i) the urethral meatus, and (ii) a proximal extent of the patient's sphincter.

41. The method of claim 40, wherein the locations of the urethral meatus and the proximal extent of the sphincter are determined by looking for a predetermined difference in pressure between adjacent pressure sensors.
